(19) United States
(12) Patent Application Publication
(10) Pub. No.: US 2023/0072946 A1
(43) Pub. Date: Mar. 9, 2023

(54) NECK MASSAGER

(71) Applicant: XIAMEN COMFIER TECHNOLOGY CO., LTD., Xiamen (CN)

(72) Inventors: Zhipeng Huang, Hangzhou (CN); Xiaying Yu, Hangzhou (CN); Shiran Huang, Hangzhou (CN)

(21) Appl. No.: 17/901,760

(22) Filed: Sep. 1, 2022

(30) Foreign Application Priority Data
Sep. 2, 2021 (CN) 202122114260.X

(51) Int. Cl. A61H 15/00; A61H 23/02

(52) U.S. Cl. CPC A61H 15/0078; A61H 23/02; A61H 2201/1611; A61H 2205/04

(57) ABSTRACT

A neck massager includes a housing, two massage heads symmetrically disposed on the housing, and two swing mechanisms for driving the two massage heads to work. The massage heads are installed on the housing. Two shock-absorbing silicone gaskets are disposed between the massage heads and the housing for installing the massage heads in a sealing manner. The swing mechanisms are disposed in the housing. The swing mechanisms are connected to the massage heads for driving the massage heads to work. Two pull straps are symmetrically disposed on the housing. The two pull straps have matching buckles to retain the housing on a user's neck.

### BACKGROUND OF THE INVENTION

### 1. Field of the Invention

The present invention relates to a massager, and more particularly to a neck massager.

### 2. Description of the Prior Art

In recent years, due to work and bad living habits, the incidence of neck diseases has shown a trend of younger people. More and more people are suffering from neck diseases. As an effective non-surgical neck care device, a neck massager becomes an essential device for many patients with neck diseases. However, the existing neck massager does not have a retaining device and only relies on a U-shaped opening to be positioned on the user's neck. When in use, the existing neck massager is not in close contact with a user's neck, so it cannot guarantee an effective massage.

### SUMMARY OF THE INVENTION

In view of the deficiencies of the prior art, the primary object of the present invention is to provide a neck massager. The neck massager has pull straps to ensure that the neck massager is retained on the user's neck and is in close contact with the user's neck, thereby achieving an effective massage.

In order to achieve the above object, the present invention adopts the following technical solutions:

A neck massager comprises a housing, two massage heads symmetrically disposed on the housing, and two swing mechanisms for driving the two massage heads to work. The massage heads are installed on the housing. Two shock-absorbing silicone gaskets are disposed between the massage heads and the housing for installing the massage heads in a sealing manner. The swing mechanisms are disposed in the housing. The swing mechanisms are connected to the massage heads for driving the massage heads to work. Two pull straps are symmetrically disposed on the housing. The two pull straps have matching buckles to retain the housing on a user's neck.

Preferably, for the massage head to have massage and heating functions and for the massage head to have a soft feel, the massage head includes a massage head upper cover, a massage head lower cover, a silicone head, a heating head, and a heating lamp panel. The massage head upper cover is in the shape of a cat's paw. The massage head lower cover is fixedly connected to the massage head upper cover. The silicone head is sleeved on the massage head upper cover. The heating lamp panel is mounted on the massage head lower cover. The heating head is disposed above the heating lamp panel. The heating head has a plurality of elastic engaging claws extending downward. The massage head upper cover has engaging grooves for engagement of the engaging claws. The silicone head has a through hole for the heating head to extend out of the silicone head.

Preferably, in order to imitate a cat's paw with massage knobs to improve the massage comfort of the massage head, a plurality of front massage knobs are disposed on a front side of an upper surface of the massage head upper cover. A plurality of rear massage knobs are disposed on a rear side of the upper surface of the massage head upper covet The front massage knobs have a height greater than that of the rear massage knobs. The heating head is located between the front massage knobs and the rear massage knobs.

Preferably, in order to drive the massage head to rotate eccentrically for expanding the massage range, the swing mechanism includes a drive box, a drive motor, a transmission gear set, an eccentric mounting seat, and a massage head drive rod. The drive motor, the transmission gear set and the eccentric mounting seat are disposed in the drive box. The drive motor is connected to the eccentric mounting seat through the transmission gear set to drive the eccentric mounting seat to rotate. One end of the massage head drive rod is connected to the eccentric mounting seat. Another end of the massage head drive rod is connected to the corresponding massage head to drive the corresponding massage head to rotate eccentrically.

Preferably, in order to drive the massage head to rotate, the transmission gear set includes a worm that is coaxially and fixedly connected to a motor shaft of the drive motor, a worm gear meshing with the worm, a first gear that is coaxially and fixedly connected to the worm gear, a second gear meshing with the first gear, a third gear that is coaxially and fixedly connected to the second gear, and a fourth gear meshing with the third gear. The eccentric mounting seat includes a cylinder that is coaxially and fixedly connected to the fourth gear. An upper side of the cylinder has an inclined mounting face. One end of a connecting shaft is connected to the cylinder. The connecting shaft is perpendicular to the inclined mounting face and located at a center of the inclined mounting face. Another end of the connecting shaft is connected to the massage head drive rod. A connecting key is disposed on the massage head drive rod. The massage head has a key groove for insertion of the connecting key.

Compared with the prior art, the present invention has the following advantages. The present invention has the pull straps to retain the housing of the neck massager on the user's neck, which ensures that the massage head is in close contact with the user's neck, so as to achieve an effective massage. The massage heads are eccentrically installed on the housing. The massage heads continuously change the massage range while rotating, so as to expand the massage range. The massage head is in the shape of a cat's paw, and the massage knobs on the massage head are arranged like a cat's claws. The front massage knobs are higher than the rear massage knobs. When the massage head is rotated, the contact depths of the front and rear massage knobs are different, giving the user different massage strengths. The silicone head is disposed on the massage head to ensure a soft touch during massage and give the user the comfort of hand massage. While the massage heads are rotated to massage the user's neck, the heating lamp panel works to warm the user's neck, so as to provide a better massage effect.

### BRIEF DESCRIPTION OF THE DRAWINGS

FIG. 1 is a perspective view of the present invention;

FIG. 2 is an exploded view of the present invention;

FIG. 3 is an exploded view of the swing mechanism of the present invention; and

FIG. 4 is an exploded view of the massage head of the present invention.

### DETAILED DESCRIPTION OF THE PREFERRED EMBODIMENTS

Embodiments of the present invention will now be described, by way of example only, with reference to the accompanying drawings.

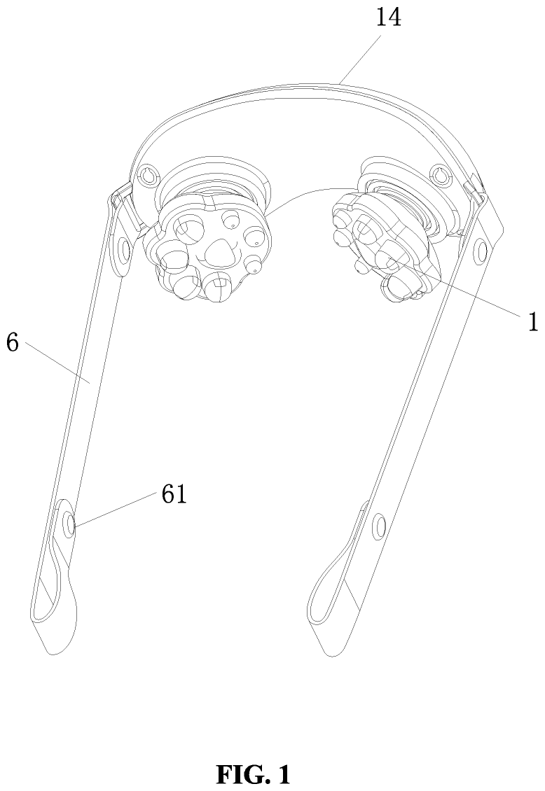

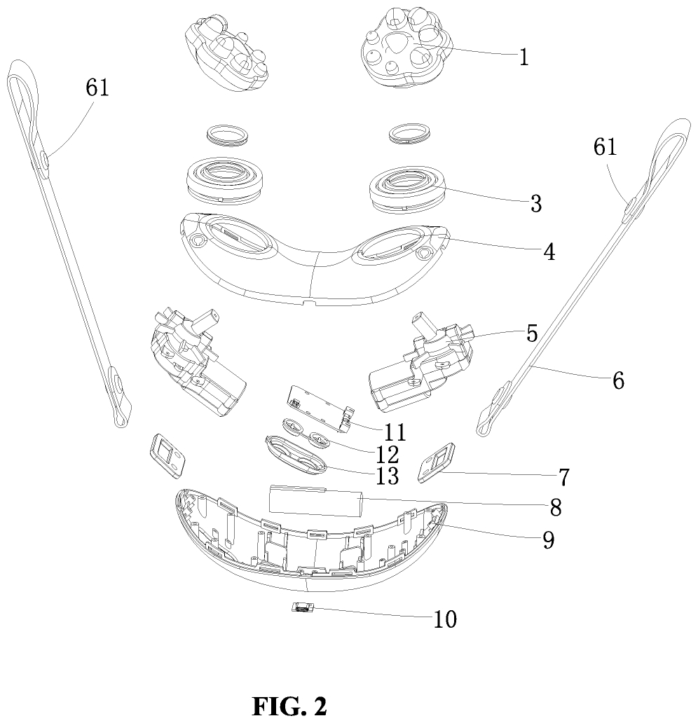

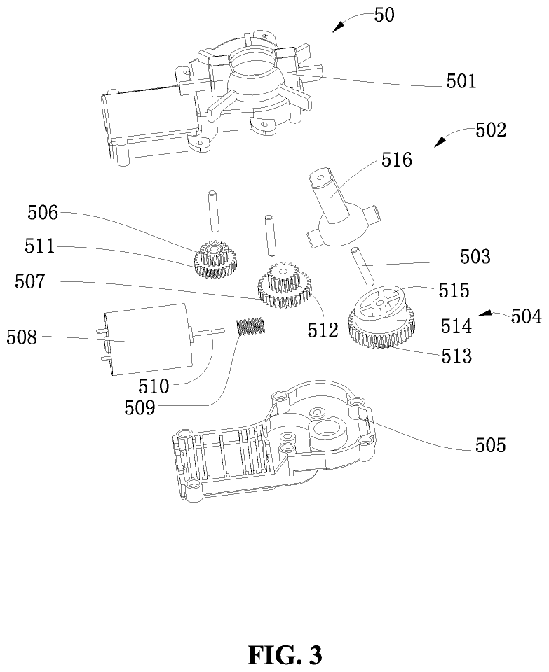

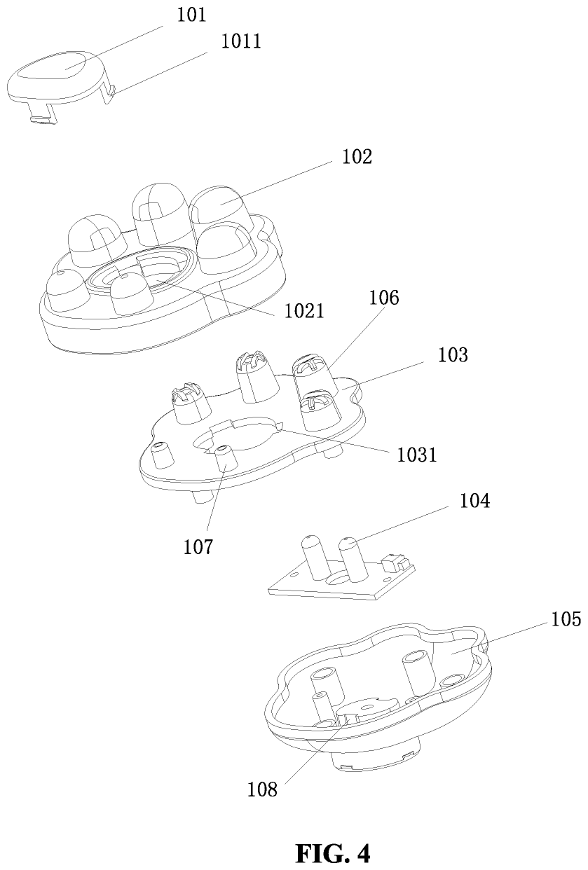

As shown in FIGS. 1-4, a neck massager according to an embodiment of the present invention comprises a housing 14, two massage heads 1 symmetrically disposed on the housing 14, and two swing mechanisms 5 for driving the two massage heads 1 to work. The massage heads 1 are installed on the housing 14. Two shock-absorbing silicone gaskets 3 are disposed between the massage heads 1 and the housing 14 for installing the massage heads 1 in a sealing manner. The shock-absorbing silicone gasket 3 also prevents the user's skin from being caught in the gap between the massage head 1 and the housing 14. The swing mechanisms 5 are disposed in the housing 14. The swing mechanisms 5 are connected to the massage heads 1 for driving the massage heads 1 to work. Two pull straps 6 are symmetrically disposed on the housing 14. The two pull straps 6 have matching buckles 61 to retain the housing 14 on a user's neck.

In this embodiment, the housing 14 includes an upper housing 4 and a lower housing 9. The upper housing 4 and the lower housing 9 are connected by screws. Two pull strap fixing members 7 are fixedly mounted between the upper housing 4 and the lower housing 9, so that the two pull straps 6 are fixed on both sides of the housing 14. A button 12 and a USB charging terminal 10 are disposed on the housing 14. The button 12 is mounted on the housing 14 through a button fixing plate 13. A circuit board 11 connected with the button 12 is disposed inside the housing 14. The circuit board 11 is connected to a battery 8 to obtain electrical energy. The circuit board 11 is connected with the swing mechanisms 5 to control the swing mechanisms 5 to work.

In this embodiment, for the massage head 1 to have massage and heating functions and for the massage head 1 to have a soft feel, the massage head 1 includes a massage head upper cover 103, a massage head lower cover 105, a silicone head 102, a heating head 101, and a heating lamp panel 104. The massage head upper cover 103 is in the shape of a cat's paw. The massage head lower cover 105 is fixedly connected to the massage head upper cover 103 by screws. The silicone head 102 is sleeved on the massage head upper cover 103. The heating lamp panel 104 is mounted on the massage head lower cover 105 by screws. The heating head 101 is disposed above the heating lamp panel 104. The heating head 101 has a plurality of elastic engaging claws 1101 extending downward. The massage head upper cover 103 has engaging grooves 1031 for engagement of the engaging claws. The silicone head 102 has a through hole 1021 for the heating head 101 to extend out of the silicone head 102.

In this embodiment, in order to imitate a cat's paw with massage knobs to improve the massage comfort of the massage head 1, a plurality of front massage knobs 106 are disposed on the front side of the upper surface of the massage head upper cover 103, and a plurality of rear massage knobs 107 are disposed on the rear side of the upper surface of the massage head upper cover 103. The shape of the silicone head 102 corresponds to the shape of the massage head upper cover 103. The height of the front massage knobs 106 is greater than the height of the rear massage knobs 107. The heating head 101 is located between the front massage knobs 106 and the rear massage knobs 107.

In this embodiment, in order to drive the massage head 1 to rotate eccentrically for expanding the massage range, the swing mechanism 5 includes a drive box 50, a drive motor 508, a transmission gear set, an eccentric mounting seat 504, and a massage head drive rod 502. The drive box 50 is composed of an upper box body 501 and a lower box body 505. The drive motor 508, the transmission gear set and the eccentric mounting seat 504 are all disposed in the drive box 50. The drive motor 508 is connected to the eccentric mounting seat 504 through the transmission gear set to drive the eccentric mounting seat 504 to rotate. One end of the massage head drive rod 502 is connected to the eccentric mounting seat 504. The other end of the massage head drive rod 502 is connected to the corresponding massage head 1 to drive the corresponding massage head 1 to rotate eccentrically.

In this embodiment, in order to drive the massage head 1 to rotate, the transmission gear set includes a worm 509 that is coaxially and fixedly connected to a motor shaft 510 of the drive motor 508, a worm gear 511 meshing with the worm 509, a first gear 506 that is coaxially and fixedly connected to the worm gear 511, a second gear 507 meshing with the first gear 506, a third gear 512 that is coaxially and fixedly connected to the second gear 507, and a fourth gear 513 meshing with the third gear 512. The eccentric mounting seat 504 includes a cylinder 514 that is coaxially and fixedly connected to the fourth gear 513. The upper side of the cylinder 514 has an inclined mounting face 515. One end of a connecting shaft 503 is connected to the cylinder 514. The connecting shaft 503 is perpendicular to the inclined mounting face 515 and located at the center of the inclined mounting face 515. The other end of the connecting shaft 503 is connected to the massage head drive rod 502. A connecting key 516 is disposed on the massage head drive rod 502. The massage head 1 has a key groove 108 for insertion of the connecting key 516.

When in use, the button 12 is pressed for the neck massager to work. The drive motor 508 outputs power to drive the worm gear 511 and the worm 509 to rotate. The worm gear 511 and the worm 509 drive the gears to rotate. Because the eccentric mounting seat 504 is coaxially and fixedly connected to the fourth gear 513, the eccentric mounting seat 504 is rotated synchronously with the fourth gear 513. The massage head drive rod 502 is mounted on the inclined mounting face 515 of the eccentric mounting seat 504, so the rotation center of the massage head drive rod 502 does not coincide with the rotation center of the eccentric mounting seat 504. The massage head 1 is rotated eccentrically to increase the massage range. The massage knobs on the massage head 1 are arranged in the shape of a cat's paw. The front massage knobs are higher than the rear massage knobs. When the massage head 1 is rotated, the contact depths of the front and rear massage knobs are different, giving the user different massage strengths. While the massage heads 1 are rotated to massage the user's neck, the heating lamp panel 104 works to wane the user's neck, so as to provide a better massage effect.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A neck massager, comprising a housing, two massage heads symmetrically disposed on the housing, and two swing mechanisms for driving the two massage heads to work; the massage heads being installed on the housing, two shock-absorbing silicone gaskets being disposed between the massage heads and the housing for installing the massage heads in a sealing manner, the swing mechanisms being disposed in the housing, the swing mechanisms being connected to the massage heads for driving the massage heads to work, two pull straps being symmetrically disposed on the housing, the two pull straps having matching buckles to retain the housing on a user's neck.

2. The neck massager as claimed in claim 1, wherein the massage head includes a massage head upper cover, a massage head lower cover, a silicone head, a heating head and a heating lamp panel, the massage head upper cover is in the shape of a cat's paw, the massage head lower cover is fixedly connected to the massage head upper cover, the silicone head is sleeved on the massage head upper cover, the heating lamp panel is mounted on the massage head lower cover, the heating head is disposed above the heating lamp panel, the heating head has a plurality of elastic engaging claws extending downward, the massage head upper cover has engaging grooves for engagement of the engaging claws, and the silicone head has a through hole for the heating head to extend out of the silicone head.

3. The neck massager as claimed in claim 2, wherein a plurality of front massage knobs are disposed on a front side of an upper surface of the massage head upper cover, a plurality of rear massage knobs are disposed on a rear side of the upper surface of the massage head upper cover, the front massage knobs have a height greater than that of the rear massage knobs, and the heating head is located between the front massage knobs and the rear massage knobs.

4. The neck massager as claimed in claim 1, wherein the swing mechanism includes a drive box, a drive motor, a transmission gear set, an eccentric mounting seat and a massage head drive rod, the drive motor, the transmission gear set and the eccentric mounting seat are disposed in the drive box, the drive motor is connected to the eccentric mounting seat through the transmission gear set to drive the eccentric mounting seat to rotate, one end of the massage head drive rod is connected to the eccentric mounting seat, and another end of the massage head drive rod is connected to the corresponding massage head to drive the corresponding massage head to rotate eccentrically.

5. The neck massager as claimed in claim 4, wherein the transmission gear set includes a worm that is coaxially and fixedly connected to a motor shaft of the drive motor, a worn gear meshing with the worm, a first gear that is coaxially and fixedly connected to the worm gear, a second gear meshing with the first gear, a third gear that is coaxially and fixedly connected to the second gear, and a fourth gear meshing with the third gear; the eccentric mounting seat includes a cylinder that is coaxially and fixedly connected to the fourth gear, an upper side of the cylinder has an inclined mounting face, one end of a connecting shaft is connected to the cylinder, the connecting shaft is perpendicular to the inclined mounting face and located at a center of the inclined mounting face, another end of the connecting shaft is connected to the massage head drive rod, a connecting key is disposed on the massage head drive rod, and the massage head has a key groove for insertion of the connecting key.
